(19) United States
(12) Patent Application Publication
(10) Pub. No.: US 2023/0238125 A1
(43) Pub. Date: Jul. 27, 2023

(54) Establishing Network Pairing

(71) Applicant: Gauss Surgical Inc., Menlo Park, CA (US)

(72) Inventors: Siddarth Satish, Redwood City, CA (US); Andrew Hosford, Idaho Falls, ID (US); Steven D. Scherf, Oakland, CA (US); Thang Sy, Menlo Park, CA (US); Charles Peterson Carroll, Berkeley, CA (US); Gaurav Retwal, Faridabad (IN)

(73) Assignee: Gauss Surgical Inc., Menlo Park, CA (US)

(21) Appl. No.: 18/106,896

(22) Filed: Feb. 7, 2023

Related U.S. Application Data
(60) Provisional application No. 63/294,565, filed on Dec. 29, 2021.

(51) Int. Cl. G16H 40/40; G16H 40/20; G06K 19/06

(52) U.S. Cl. CPC G16H 40/40; G16H 40/20; G06K 19/06009

(57) ABSTRACT

A machine causes capture of an image that depicts an optically readable code exhibited by a measurement device that corresponds to a scheduled medical procedure. The machine then determines, based on the optically readable code depicted by the captured image, a hardware identifier of a network interface of the measurement device and a location identifier of the measurement device. The machine next identifies the medical procedure based on the location identifier determined based on the optically readable code exhibited by the measurement device. The machine establishes a network connection with the measurement device based on the determined hardware identifier of the network interface of the measurement device. The established network connection may provide access to measurement data generated by the measurement device that corresponds to the scheduled medical procedure identified based on the location identifier of the measurement device.

### CROSS REFERENCE TO RELATED APPLICATION

This present application claims priority to and all the benefits of U.S. Provisional Patent Application No. 63/294,565, filed on Dec. 29, 2021, the entire contents of which are hereby incorporated by reference.

### TECHNICAL FIELD

The subject matter disclosed herein generally relates to the technical field of special-purpose machines that facilitate establishment of network communications between or among devices (e.g., mobile devices, measuring devices, or other devices), including software-configured computerized variants of such special-purpose machines and improvements to such variants, and to the technologies by which such special-purpose machines become improved compared to other special-purpose machines that facilitate establishing network communications between or among devices. Specifically, the present disclosure addresses systems and methods to facilitate establishing a network pairing between devices.

### BACKGROUND

A machine (e.g., a device, a computer, or other machine) may be configured to interact with one or more users by responding to user-submitted requests, commands, or other user input, via a user interface, such as a graphical user interface. The machine may be configured to establish one or more network connections for network communications with one or more other machines. The establishment of a given network connection (e.g., a network pairing of the machine with another machine) may be in response to user input or may be automatically performed, such as in response to fulfillment of a triggering condition (e.g., a detection that the two machines to be paired are within a threshold distance of each other). Once established, the network connection facilitates communication of data between the connected machines.

### BRIEF DESCRIPTION OF THE DRAWINGS

Some embodiments are illustrated by way of example and not limitation in the figures of the accompanying drawings.

FIG. 1 is a network diagram illustrating a network environment suitable for establishing a network pairing, according to some example embodiments.

FIG. 2 is a block diagram illustrating components of a machine (e.g., a mobile device) suitable for establishing a network pairing, according to some example embodiments.

FIGS. 3-5 are flowcharts illustrating operations of a device (e.g., a mobile device) in performing a method of establishing a network pairing, according to some example embodiments.

FIG. 6 is a block diagram illustrating components of a machine, according to some example embodiments, able to read instructions from a machine-readable medium and perform any one or more of the methodologies discussed herein.

### DETAILED DESCRIPTION

Example methods (e.g., algorithms) facilitate establishing one or more network pairings, and example systems (e.g., special-purpose machines configured by special-purpose software) are configured to facilitate establishing one or more network pairings. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

A machine, such as a mobile device, is configures (e.g., by suitably programmed hardware, software, or both) to establish a network pairing with another device, such as a measurement device (e.g., a scale for weighing one or more items). In accordance with some examples of the methods and systems described herein, such a suitably configured machine causes capture of an image that depicts an optically readable code (e.g., a machine-readable optical code, such as a quick response (QR) code or a barcode) exhibited by a measurement device. The machine then, based on the captured image, determines a hardware identifier of the measurement device. Based on the determined hardware identifier, the machine accordingly establishes a network connection (e.g., a wireless network connection, such as a BLUETOOTH® wireless network pairing).

In accordance with certain other examples of the methods and systems described herein, such a suitably configured machine causes capture of an image that depicts an optically readable code exhibited by a measurement device that corresponds to a scheduled medical procedure. The machine then determines, based on the optically readable code depicted by the captured image, a hardware identifier of a network interface of the measurement device and a location identifier of the measurement device. The machines next identifies (e.g., by performing one or more database lookups) the medical procedure based on the location identifier determined based on the optically readable code exhibited by the measurement device. The machine accordingly establishes a network connection (e.g., a wireless network connection, such as a BLUETOOTH® wireless network pairing) with the measurement device based on the determined hardware identifier of the network interface of the measurement device, and the established network connection may provide access to measurement data generated by the measurement device that corresponds to the scheduled medical procedure identified based on the location identifier of the measurement device. Further details are discussed below for various example embodiments of the systems and method described herein

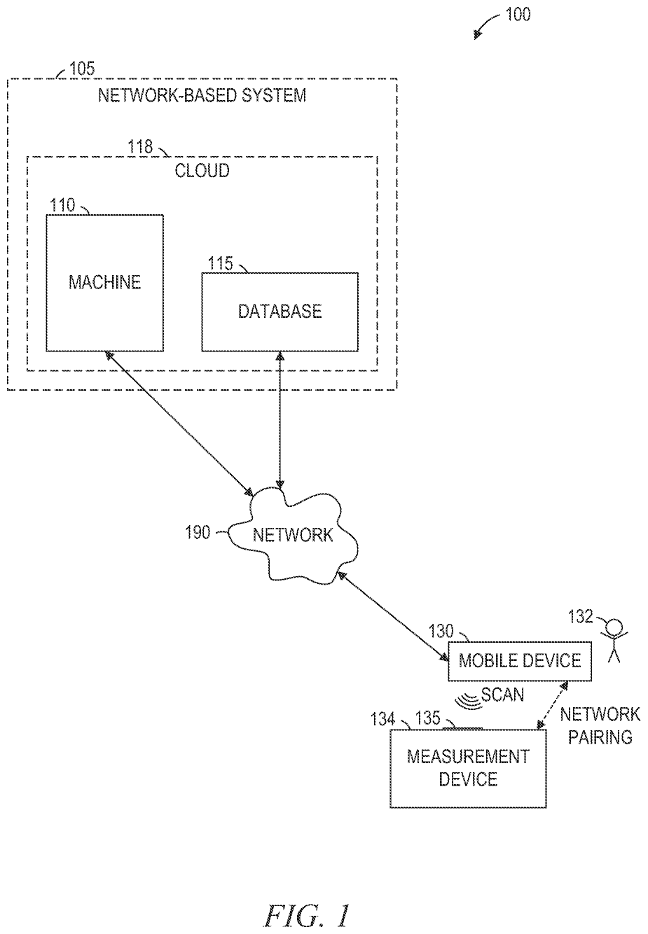

FIG. 1 is a network diagram illustrating a network environment 100 suitable for establishing a network pairing, according to some example embodiments. The network environment 100 includes a machine 110 (e.g., a server machine or cloud of server machines), a database 115 (e.g., a database server machine), and a mobile device 130 (e.g., a smartphone), all communicatively coupled to each other via a network 190. The mobile device 130 is shown as being in position (e.g., within line of sight) to scan and read an optically readable code 135 (e.g., a QR code) that is being exhibited (e.g., displayed) by a measurement device 134 (e.g., a scale for weighing one or more items). The optically readable code 135 may be or include one or more optically machine-readable indicia. In some example embodiments, the optically machine-readable indicia are also human-readable (e.g., text). In other example embodiments, the optically machine-readable indicia are not human-readable (e.g., a barcode or a QR code). According to the systems and methods described herein, the mobile device 130 is able to establish (e.g., manually, or automatically) a network connection to (e.g., a network pairing with) the measurement device 134 (e.g., based on the mobile device 130 scanning the machine-readable code exhibited by the measurement device 134).

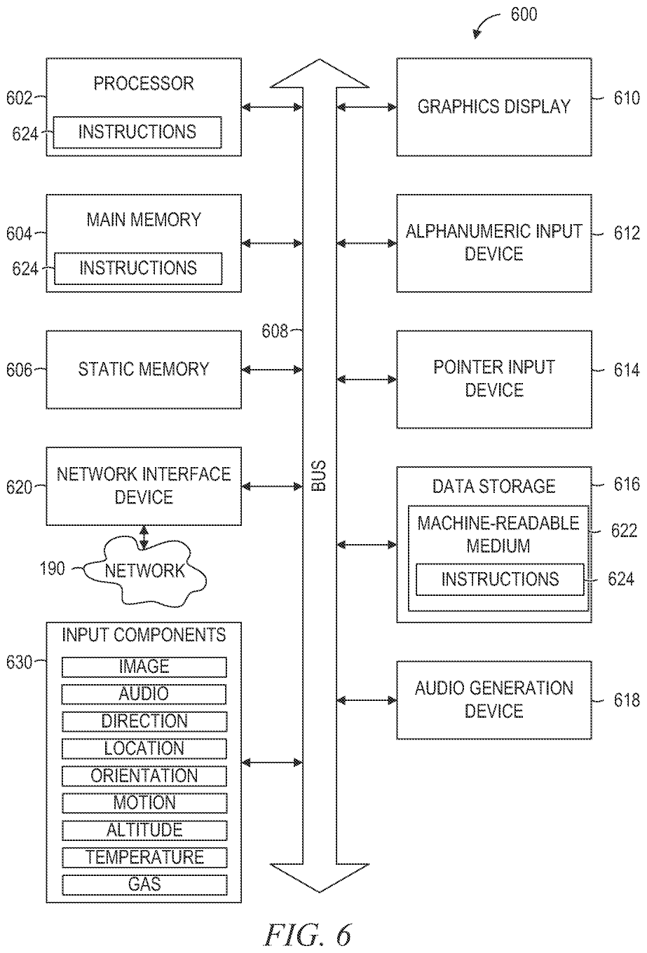

The machine 110, with or without the database 115, may form all or part of a cloud 118 (e.g., a geographically distributed set of multiple machines configured to function as a single server), which may form all or part of a network-based system 105 (e.g., a cloud-based server system configured to provide one or more network-based services to the mobile device 130). The machine 110, the database 115, the mobile device 130, and the measurement device 134 may each be implemented in a special-purpose (e.g., specialized) computer system, in whole or in part, as described below with respect to FIG. 6.

Also shown in FIG. 1 is a user 132. The user 132 may be a human user (e.g., a human being), a machine user (e.g., a computer configured by a software program to interact with the mobile device 130), or any suitable combination thereof (e.g., a human assisted by a machine or a machine supervised by a human). The user 132 is associated with the mobile device 130 (e.g., as well as the measurement device 134) and may be a user of the mobile device 130 (e.g., as well as a user of the measurement device 134). For example, the mobile device 130 may be a desktop computer, a vehicle computer, a home media system (e.g., a home theater system or other home entertainment system), a tablet computer, a navigational device, a portable media device, a smart phone, or a wearable device (e.g., a smart watch, smart glasses, smart clothing, or smart jewelry) belonging to the user 132.

Any of the systems or machines (e.g., databases and devices) shown in FIG. 1 may be, include, or otherwise be implemented in a special-purpose (e.g., specialized or otherwise non-conventional and non-generic) computer that has been modified to perform one or more of the functions described herein for that system or machine (e.g., configured or programmed by special-purpose software, such as one or more software modules of a special-purpose application, operating system, firmware, middleware, or other software program). For example, a special-purpose computer system able to implement any one or more of the methodologies described herein is discussed below with respect to FIG. 6, and such a special-purpose computer may accordingly be a means for performing any one or more of the methodologies discussed herein. Within the technical field of such special-purpose computers, a special-purpose computer that has been specially modified (e.g., configured by special-purpose software) by the structures discussed herein to perform the functions discussed herein is technically improved compared to other special-purpose computers that lack the structures discussed herein or are otherwise unable to perform the functions discussed herein. Accordingly, a special-purpose machine configured according to the systems and methods discussed herein provides an improvement to the technology of similar special-purpose machines.

As used herein, a “database” is a data storage resource and may store data structured in any of various ways, for example, as a text file, a table, a spreadsheet, a relational database (e.g., an object-relational database), a triple store, a hierarchical data store, a document database, a graph database, key-value pairs, or any suitable combination thereof. Moreover, any two or more of the systems or machines illustrated in FIG. 1 may be combined into a single system or machine, and the functions described herein for any single system or machine may be subdivided among multiple systems or machines.

The network 190 may be any network that enables communication between or among systems, machines, databases, and devices (e.g., between the machine 110 and the mobile device 130). Accordingly, the network 190 may be a wired network, a wireless network (e.g., a mobile or cellular network), or any suitable combination thereof. The network 190 may include one or more portions that constitute a private network, a public network (e.g., the Internet), or any suitable combination thereof. Accordingly, the network 190 may include one or more portions that incorporate a local area network (LAN), a wide area network (WAN), the Internet, a mobile telephone network (e.g., a cellular network), a wired telephone network (e.g., a plain old telephone service (POTS) network), a wireless data network (e.g., a WiFi network or WiMax network), or any suitable combination thereof. Any one or more portions of the network 190 may communicate information via a transmission medium. As used herein, “transmission medium” refers to any intangible (e.g., transitory) medium that is capable of communicating (e.g., transmitting) instructions for execution by a machine (e.g., by one or more processors of such a machine), and includes digital or analog communication signals or other intangible media to facilitate communication of such software.

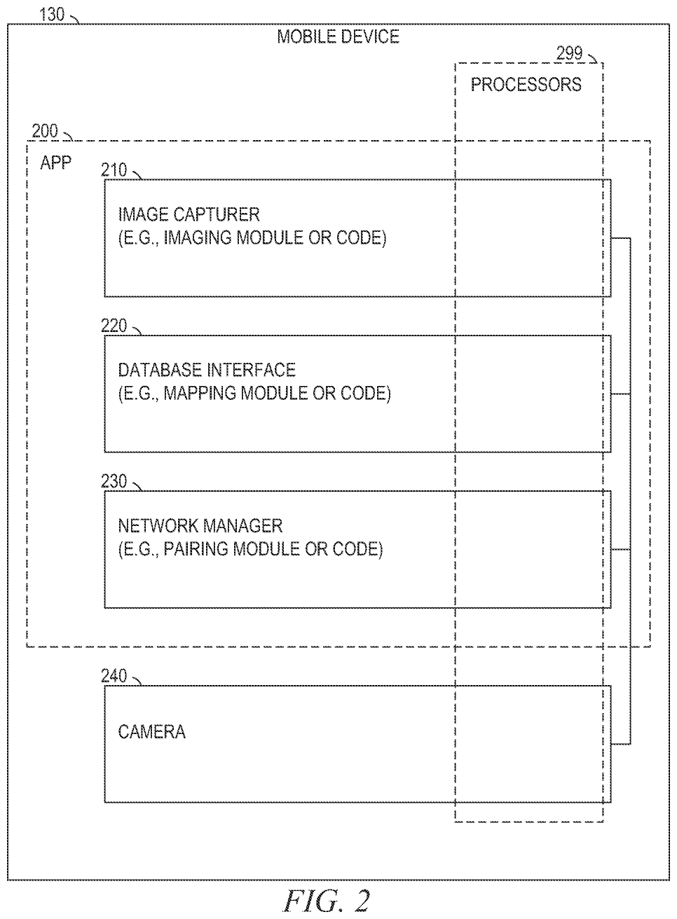

FIG. 2 is a block diagram illustrating components of the mobile device 130 (e.g., a smartphone) suitable for establishing a network pairing, according to some example embodiments. The mobile device 130 is shown as including an image capturer 210 (e.g., an imaging module, similarly suitable software code, or any suitable combination thereof), a database interface 220 (e.g., a mapping module, similarly suitable software code, or any suitable combination thereof), a network manager 230 (e.g., a pairing module, similarly suitable software code, or any suitable combination thereof), and a camera 240 (e.g., an optical light camera), all configured to communicate with each other (e.g., via a bus, shared memory, or a switch).

As shown in FIG. 2, the image capturer 210, the database interface 220, and the network manager 230 may form all or part of an app 200 (e.g., a mobile app) that is stored (e.g., installed) on the mobile device 130 (e.g., responsive to or otherwise as a result of data being received from the machine 110 or the database 115, via the network 190). Furthermore, one or more processors 299 (e.g., hardware processors, digital processors, or any suitable combination thereof) may be included (e.g., temporarily or permanently) in the app 200, the image capturer 210, the database interface 220, the network manager 230, or any suitable combination thereof.

Any one or more of the components (e.g., modules) described herein may be implemented using hardware alone (e.g., one or more of the processors 299) or a combination of hardware and software. For example, any component described herein may physically include an arrangement of one or more of the processors 299 (e.g., a subset of or among the processors 299) configured to perform the operations described herein for that component. As another example, any component described herein may include software, hardware, or both, that configure an arrangement of one or more of the processors 299 to perform the operations described herein for that component. Accordingly, different components described herein may include and configure different arrangements of the processors 299 at different points in time or a single arrangement of the processors 299 at different points in time. Each component (e.g., module) described herein is an example of a means for performing the operations described herein for that component. Moreover, any two or more components described herein may be combined into a single component, and the functions described herein for a single component may be subdivided among multiple components. Furthermore, according to various example embodiments, components described herein as being implemented within a single system or machine (e.g., a single device) may be distributed across multiple systems or machines (e.g., multiple devices).

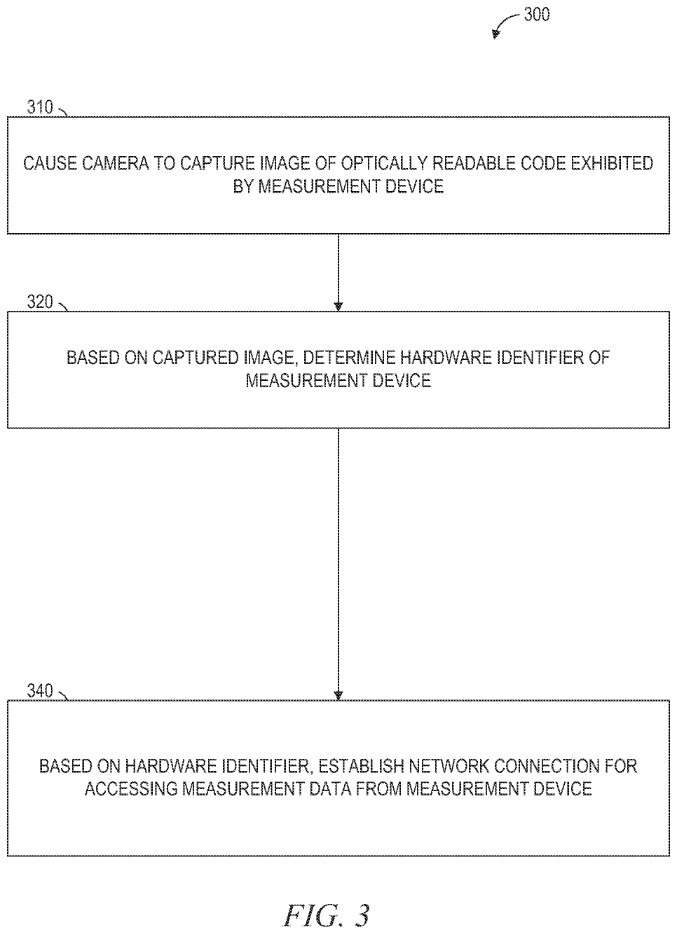

FIGS. 3-5 are flowcharts illustrating operations of the mobile device 130 (e.g., a smartphone, a smartwatch, a smart badge, smart glasses, or other mobile device) in performing a method 300 of establishing a network pairing, according to some example embodiments. Operations in the method 300 may be performed by the mobile device 130, using components (e.g., modules) described above with respect to FIG. 2, using one or more processors (e.g., microprocessors or other hardware processors), or using any suitable combination thereof.

As shown in FIG. 3, some example embodiments of the method 300 include (e.g., at least) operations 310, 320, and 340.

In operation 310 of such example embodiments, the image capturer 210 causes (e.g., via command, request, or other control signal) the camera 240 of the mobile device 130 to capture an image that depicts the optically readable code 135 (e.g., a QR code) being exhibited (e.g., displayed) by the measurement device 134. The measurement device 134 may exhibit the optically readable code 135 on a placard, a sticker, a label, a display screen, or any suitable combination thereof, and in operation 310, the camera 240 may be caused by the image capturer 210 to take a photographic image of the optically readable code 135 exhibited on the placard, the sticker, the label, the display screen, or any suitable combination thereof.

In operation 320 of such example embodiments, the database interface 220 determines a hardware identifier of the measurement device 134, based on the image caused to be captured in operation 310. The hardware identifier of the measurement device 134 may be determined by accessing a database (e.g., in the app 200, in the database 115, or any suitable combination thereof) that maps the hardware identifier of the measurement device 134 directly or indirectly to all or part of the information encoded in the optically readable code 135. In various example embodiments, the hardware identifier is or includes a hardware address of a network interface (e.g., a BLUETOOTH® wireless network interface) of the measurement device 134.

In operation 340 of such example embodiments, the network manager 230 establishes a network connection (e.g., a BLUETOOTH® wireless network pairing) between the mobile device 130 and the measurement device 134, based on the hardware identifier of the measurement device 134, as determined in operation 320. The resulting established network connection may accordingly enable the mobile device 130 (e.g., via the app 200) to access data (e.g., measurement data) from the measurement device 134, such as measurement data generated by the measurement device 134, normalized by the measurement device 134, stored by the measurement device 134, or any suitable combination thereof.

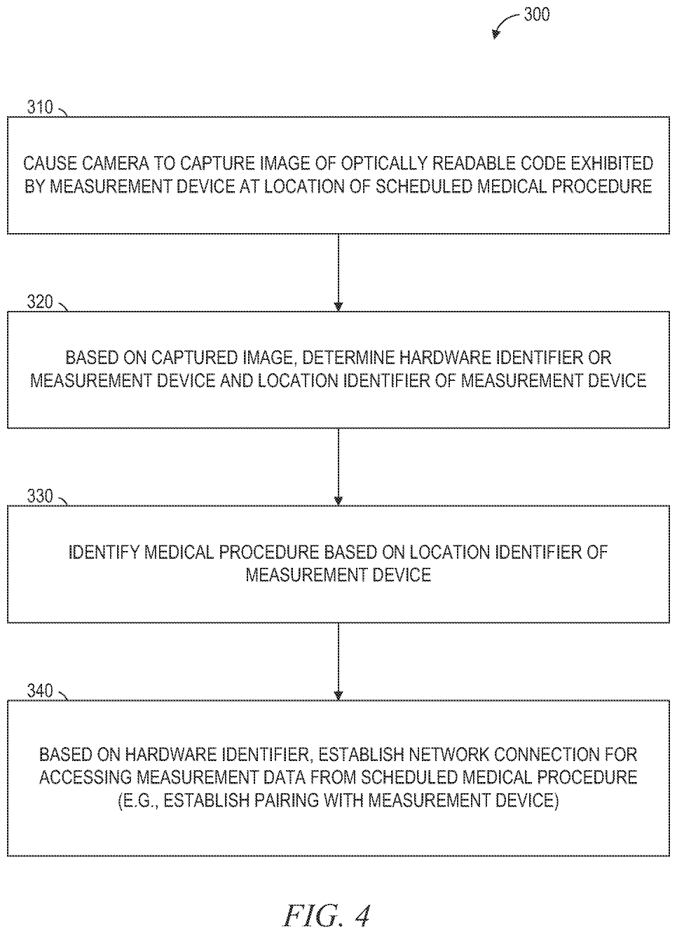

As shown in FIG. 4, certain example embodiments of the method 300 include (e.g., at least) operations 310, 320, 330, and 340.

In operation 310 of such example embodiments, the image capturer 210 causes (e.g., via command, request, or other control signal) the camera 240 of the mobile device 130 to capture an image that depicts the optically readable code 135 (e.g., a QR code) being exhibited (e.g., displayed) by the measurement device 134. The measurement device 134 may be positioned, placed, or otherwise located at a location (e.g., in an operating room or other room of a building, such as a hospital or other medical building) at which a procedure (e.g., a medical procedure, such as a surgery) is scheduled to be performed. As noted above, the measurement device 134 may exhibit the optically readable code 135 on a placard, a sticker, a label, a display screen, or any suitable combination thereof. Accordingly, in operation 310, the camera 240 may be caused by the image capturer 210 to take a photographic image of the optically readable code 135 exhibited on the placard, the sticker, the label, the display screen, or any suitable combination thereof, of the measurement device 134 at the location of the scheduled procedure.

In operation 320 of such example embodiments, the database interface 220 determines a hardware identifier of the measurement device 134, based on the image caused to be captured in operation 310, as noted above. In addition, during performance of operation 320, the database interface 220 may determine a location identifier of the measurement device 134. The determined location identifier may correspond to, and thus identify, the location of the measurement device 134 (e.g., in the operating room), which may also be the location at which the procedure is scheduled to be performed.

As noted above, the hardware identifier of the measurement device 134 may be determined by accessing a database (e.g., in the app 200, in the database 115, or any suitable combination thereof) that maps the hardware identifier of the measurement device 134 directly or indirectly to all or part of the information encoded in the optically readable code 135, and in various example embodiments, the hardware identifier is or includes a hardware address of a network interface (e.g., a BLUETOOTH® wireless network interface) of the measurement device 134. Similarly, the location identifier of the measurement device 134 may be determined by accessing a database (e.g., in the app 200, in the database 115, or any suitable combination thereof) that maps the location identifier of the measurement device 134 directly or indirectly to all or part of the information encoded in the optically readable code 135. Thus, in various example embodiments, the location identifier of the measurement device 134 is or includes an address (e.g., a room number, a floor number, a building number, a street address, a city name, or any suitable combination thereof) that corresponds to the location of the measurement device 134.

In operation 330 of such example embodiments, the database interface 220 identifies (e.g., via lookup) the procedure (e.g., the medical procedure) scheduled to be performed at or in the location of the measurement device 134. The identifying of the procedure may be based on the determined location identifier of the measurement device 134, a current time of day, a current date, or any suitable combination thereof. The procedure may be identified by accessing a database (e.g., in the app 200, in the database 115, or any suitable combination thereof) that maps the location identifier of the measurement device 134 directly or indirectly to a procedure identifier of the procedure scheduled to be performed at or in the location of the measurement device 134. In various example embodiments, the procedure identifier is or includes a procedure code, which may be unique among an available set of procedure codes, for identifying the procedure among other procedures available to be performed.

In operation 340 of such example embodiments, the network manager 230 establishes a network connection (e.g., a BLUETOOTH® wireless network pairing) between the mobile device 130 and the measurement device 134, based on the hardware identifier of the measurement device 134, as determined in operation 320, and optionally based on the location identifier of the measurement device 134, as may have been determined in operation 320. The resulting established network connection may accordingly enable the mobile device 130 (e.g., via the app 200) to access data (e.g., measurement data that corresponds to the scheduled procedure) from the measurement device 134, such as measurement data that corresponds to the scheduled procedure. Such measurement data from the scheduled procedure may be generated by the measurement device 134 during the identified procedure scheduled to be performed at or in the location that corresponds to the determined location identifier of the measurement device 134, normalized by the measurement device 134 during the identified procedure scheduled to be performed at or in the location that corresponds to the determined location identifier of the measurement device 134, stored by the measurement device 134 during the identified procedure scheduled to be performed at or in the location that corresponds to the determined location identifier of the measurement device 134, or any suitable combination thereof.

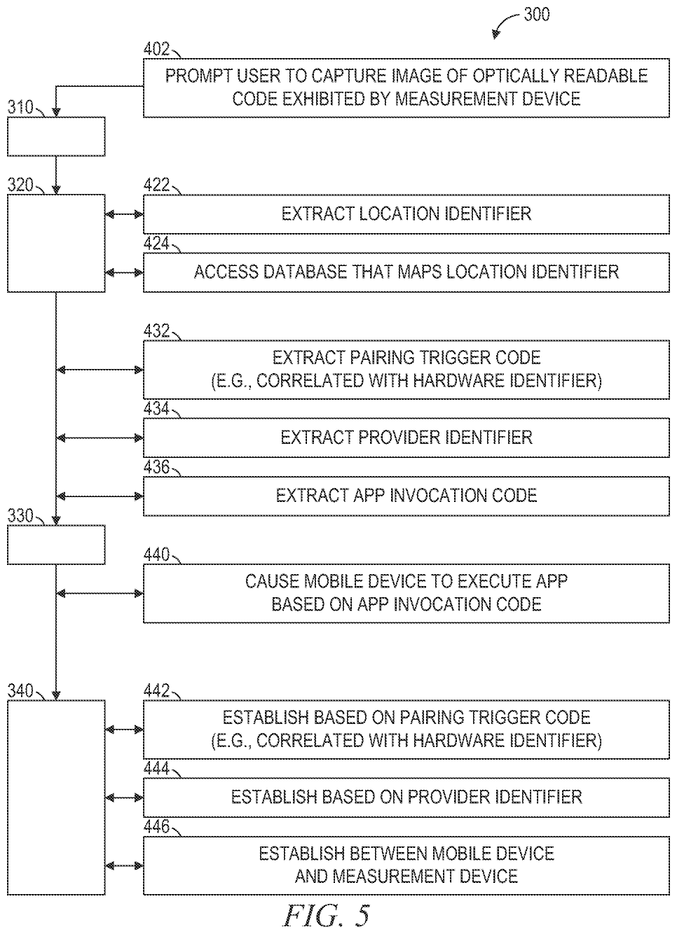

As shown in FIG. 5, various example embodiments of the method 300 include one or more of operations 402, 422, 424, 432, 434,436, 440, 442, 444, and 446 (e.g., in addition to one or more of the operations 310, 320, 330, and 340, as described above).

Operation 402 may be performed prior to operation 310. In operation 402, the app 200 prompts (e.g., by presenting a dialog box or an alert) the user 132 of the mobile device 130 to initiate the establishing of the network connection with the measurement device 134 by scanning the optically readable code 135 exhibited by the measurement device 134 via the camera 240 of the mobile device 130.

One or more of operations 422 and 424 may be performed as part (e.g., a precursor task, a subroutine, or a portion) of operation 320, in which the database interface 220 determines the hardware identifier of the measurement device 134 and may determine the location identifier of the measurement device 134.

In operation 422, as part of determining the location identifier of the measurement device 134, the database interface 220 extracts the location identifier from the optically readable code 135 exhibited by the measurement device 134. For example, the location identifier may be encoded in some or all of the optically readable code 135, and the database interface 220 may extract the location identifier by decoding some or all of the optically readable code 135.

In operation 424, as part of determining the location identifier of the measurement device 134, the database interface 220 accesses a database (e.g., in the app 200, in the database 115, or any suitable combination thereof) that maps the location identifier to an intermediate identifier within the optically readable code 135. For example, the intermediate identifier may be encoded in some or all of the optically readable code 135, and the database interface 220 may decode the intermediate identifier and perform a database lookup to determine the location identifier based on the location identifier being mapped by the database (e.g., in the app 200 or in the database 115) to the intermediate identifier.

In operation 432, the database interface 220 extracts a pairing trigger code from the optically readable code 135 depicted by the image captured in operation 310. For example, the pairing code may be encoded in some or all of the optically readable code 135, and the database interface 220 may extract the location identifier by decoding some or all of the optically readable code 135. As another example, an intermediate code may be encoded in some or all of the optically readable code 135, and the database interface 220 may decode the intermediate code, access a database (e.g., in the app 200, in the database 115, or any suitable combination thereof) that maps the intermediate code to the pairing code (e.g., as well as to the hardware identifier of the measurement device 134), and perform a database lookup to determine the pairing code based on the pairing code being mapped by the database to the intermediate code.

Correspondingly, in example embodiments that include operation 432, performance of operation 340, in which the network manager 230 establishes the network connection between the mobile device 130 and the measurement device 134, includes performance of operation 442. In operation 442, the network manager 230 establishes the network connection based on (e.g., in response to) the extracting of the pairing trigger code from the optically readable code 135 exhibited by the measurement device 134.

In some example embodiments, the extracting of the pairing trigger code in operation 432 (e.g., from the optically readable code from which the hardware identifier is extracted) also correlates the hardware identifier of the measurement device 134 with the extracted pairing trigger code. In such example embodiments, the establishing of the network connection in operation 442 (e.g., as part of operation 340) is based on (e.g., in response to) the extracted hardware identifier being correlated with the extracted pairing trigger code.

In operation 434, the database interface 220 extracts a provider identifier from the optically readable code 135 depicted by the image captured in operation 310, where the provider identifier identifies a provider of the measurement device 134. For example, the provider identifier of the provider of the measurement device 134 may be encoded in some or all of the optically readable code 135, and the database interface 220 may extract the provider identifier by decoding some or all of the optically readable code 135. As another example, an intermediate code may be encoded in some or all of the optically readable code 135, and the database interface 220 may decode the intermediate code, access a database (e.g., in the app 200, in the database 115, or any suitable combination thereof) that maps the intermediate code to the provider identifier of the provider of the measurement device 134 (e.g., as well as to the hardware identifier of the measurement device 134), and perform a database lookup to determine the provider identifier based on the provider identifier being mapped by the database to the intermediate code.

Correspondingly, in example embodiments that include operation 434, performance of operation 340, in which the network manager 230 establishes the network connection between the mobile device 130 and the measurement device 134, includes performance of operation 444. In operation 444, the network manager 230 establishes the network connection based on (e.g., in response to) the extracting of the provider identifier of the provider of the measurement device 134 from the optically readable code 135 exhibited by the measurement device 134.

In operation 436, the database interface 220 extracts an app invocation code that corresponds to the app 200 installed on the mobile device 130, and the app invocation code may be extracted from the optically readable code 135 depicted by the image captured in operation 310. The app invocation code for the app 200 may be or include a command, request, or other control signal to invoke execution (e.g., launch) of the app 200 on the mobile device 130. For example, the app invocation code for the app 200 may be encoded in some or all of the optically readable code 135, and the database interface 220 may extract the app invocation code by decoding some or all of the optically readable code 135. As another example, an intermediate code may be encoded in some or all of the optically readable code 135, and the database interface 220 may decode the intermediate code, access a database (e.g., in the app 200, in the database 115, or any suitable combination thereof) that maps the intermediate code to the app invocation code for the app 200 (e.g., as well as to the hardware identifier of the measurement device 134), and perform a database lookup to determine the app invocation code based on the app invocation code being mapped by the database to the intermediate code.

Correspondingly, in example embodiments that include operation 436, operation 440 may be performed based on (e.g., in response to) performance of operation 436. In operation 440, the processors 299 of the mobile device 130 execute or otherwise cause the mobile device 130 to execute the app 200 that corresponds to the app invocation code (e.g., based on the extracting of the app invocation code from the optically readable code 135 exhibited by the measurement device 134).

In certain example embodiments, performance of operation 340, in which the network manager 230 establishes the network connection between the mobile device 130 and the measurement device 134, is based on (e.g., in response to) the execution of the app 200 based on the app invocation code extracted in operation 436. For example, in such example embodiments, operation 446 may be performed as part (e.g., a precursor task, a subroutine, or a portion) of operation 340. In operation 446, the network manager 230 establishes the network connection with the measurement device 134 by establishing a network connection between whichever mobile device (e.g., mobile device 130) was caused to execute the app 200 via the app invocation code and whichever measurement device (e.g., measurement device 134) exhibited the optically readable code 135 depicted in the image captured in operation 310. In situations where there are multiple mobile devices (e.g., similar to the mobile device 130), multiple measurement devices (e.g., similar to the measurement device 134), or both, within range of each other and able to establish network connections between or among each other, performance of operation 446 may be helpful to avoid incorrect or inappropriate network pairings, ensure correct and appropriate network pairings, or both.

According to various example embodiments, one or more of the methodologies described herein may facilitate establishing one or more network connections (e.g., one or more network pairings). Moreover, one or more of the methodologies described herein may facilitate speed, convenience, accuracy, and reliability in establishing network connections, including avoiding incorrect or inappropriate network pairings, ensuring correct and appropriate network pairings, or both. Hence, one or more of the methodologies described herein may facilitate speed, convenience, accuracy, and reliability in data access, data provision, or both, with corresponding benefits to the outcomes of procedures (e.g., medical procedures) in which such data is generated, normalized, stored, provided, or any suitable combination thereof, compared to capabilities of pre-existing systems and methods.

When these effects are considered in aggregate, one or more of the methodologies described herein may obviate a need for certain efforts or resources that otherwise would be involved in establishing one or more network connections, especially where speed, convenience, accuracy, reliability, or any suitable combination thereof, are beneficial. Efforts expended by a user in establishing one or more network connections may be reduced by use of (e.g., reliance upon) a special-purpose machine that implements one or more of the methodologies described herein. Computing resources used by one or more systems or machines (e.g., within the network environment 100) may similarly be reduced (e.g., compared to systems or machines that lack the structures discussed herein or are otherwise unable to perform the functions discussed herein). Examples of such computing resources include processor cycles, network traffic, computational capacity, main memory usage, graphics rendering capacity, graphics memory usage, data storage capacity, power consumption, and cooling capacity.

FIG. 6 is a block diagram illustrating components of a machine 600, according to some example embodiments, able to read instructions 624 from a machine-readable medium 622 (e.g., a non-transitory machine-readable medium, a machine-readable storage medium, a computer-readable storage medium, or any suitable combination thereof) and perform any one or more of the methodologies discussed herein, in whole or in part. Specifically, FIG. 6 shows the machine 600 in the example form of a computer system (e.g., a computer) within which the instructions 624 (e.g., software, a program, an application, an applet, an app, or other executable code) for causing the machine 600 to perform any one or more of the methodologies discussed herein may be executed, in whole or in part.

In alternative embodiments, the machine 600 operates as a standalone device or may be communicatively coupled (e.g., networked) to other machines. In a networked deployment, the machine 600 may operate in the capacity of a server machine or a client machine in a server-client network environment, or as a peer machine in a distributed (e.g., peer-to-peer) network environment. The machine 600 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 624, sequentially or otherwise, that specify actions to be taken by that machine. Further, while only a single machine is illustrated, the term “machine” shall also be taken to include any collection of machines that individually or jointly execute the instructions 624 to perform all or part of any one or more of the methodologies discussed herein.

The machine 600 includes a processor 602 (e.g., one or more central processing units (CPUs), one or more graphics processing units (GPUs), one or more digital signal processors (DSPs), one or more application specific integrated circuits (ASICs), one or more radio-frequency integrated circuits (RFICs), or any suitable combination thereof), a main memory 604, and a static memory 606, which are configured to communicate with each other via a bus 608. The processor 602 contains solid-state digital microcircuits (e.g., electronic, optical, or both) that are configurable, temporarily or permanently, by some or all of the instructions 624 such that the processor 602 is configurable to perform any one or more of the methodologies described herein, in whole or in part. For example, a set of one or more microcircuits of the processor 602 may be configurable to execute one or more modules (e.g., software modules) described herein. In some example embodiments, the processor 602 is a multicore CPU (e.g., a dual-core CPU, a quad-core CPU, an 8-core CPU, or a 128-core CPU) within which each of multiple cores behaves as a separate processor that is able to perform any one or more of the methodologies discussed herein, in whole or in part. Although the beneficial effects described herein may be provided by the machine 600 with at least the processor 602, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 600 may further include a graphics display 610 (e.g., a plasma display panel (PDP), a light emitting diode (LED) display, a liquid crystal display (LCD), a projector, a cathode ray tube (CRT), or any other display capable of displaying graphics or video). The machine 600 may also include an alphanumeric input device 612 (e.g., a keyboard or keypad), a pointer input device 614 (e.g., a mouse, a touchpad, a touchscreen, a trackball, a joystick, a stylus, a motion sensor, an eye tracking device, a data glove, or other pointing instrument), a data storage 616, an audio generation device 618 (e.g., a sound card, an amplifier, a speaker, a headphone jack, or any suitable combination thereof), and a network interface device 620.

The data storage 616 (e.g., a data storage device) includes the machine-readable medium 622 (e.g., a tangible and non-transitory machine-readable storage medium) on which are stored the instructions 624 embodying any one or more of the methodologies or functions described herein. The instructions 624 may also reside, completely or at least partially, within the main memory 604, within the static memory 606, within the processor 602 (e.g., within the processor's cache memory), or any suitable combination thereof, before or during execution thereof by the machine 600. Accordingly, the main memory 604, the static memory 606, and the processor 602 may be considered machine-readable media (e.g., tangible and non-transitory machine-readable media). The instructions 624 may be transmitted or received over the network 190 via the network interface device 620. For example, the network interface device 620 may communicate the instructions 624 using any one or more transfer protocols (e.g., hypertext transfer protocol (HTTP)).

In some example embodiments, the machine 600 may be a portable computing device (e.g., a smart phone, a tablet computer, or a wearable device) and may have one or more additional input components 630 (e.g., sensors or gauges). Examples of such input components 630 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component (e.g., a gas sensor). Input data gathered by any one or more of these input components 630 may be accessible and available for use by any of the modules described herein (e.g., with suitable privacy notifications and protections, such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 622 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 624 for execution by the machine 600, such that the instructions 624, when executed by one or more processors of the machine 600 (e.g., processor 602), cause the machine 600 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 624 for execution by the machine 600 can be communicated via a carrier medium (e.g., a machine-readable carrier medium). Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 624).

Certain example embodiments are described herein as including modules. Modules may constitute software modules (e.g., code stored or otherwise embodied in a machine-readable medium or in a transmission medium), hardware modules, or any suitable combination thereof. A “hardware module” is a tangible (e.g., non-transitory) physical component (e.g., a set of one or more processors) capable of performing certain operations and may be configured or arranged in a certain physical manner. In various example embodiments, one or more computer systems or one or more hardware modules thereof may be configured by software (e.g., an application or portion thereof) as a hardware module that operates to perform operations described herein for that module.

In some example embodiments, a hardware module may be implemented mechanically, electronically, hydraulically, or any suitable combination thereof. For example, a hardware module may include dedicated circuitry or logic that is permanently configured to perform certain operations. A hardware module may be or include a special-purpose processor, such as a field programmable gate array (FPGA) or an ASIC. A hardware module may also include programmable logic or circuitry that is temporarily configured by software to perform certain operations. As an example, a hardware module may include software encompassed within a CPU or other programmable processor. It will be appreciated that the decision to implement a hardware module mechanically, hydraulically, in dedicated and permanently configured circuitry, or in temporarily configured circuitry (e.g., configured by software) may be driven by cost and time considerations.

Accordingly, the phrase “hardware module” should be understood to encompass a tangible entity that may be physically constructed, permanently configured (e.g., hardwired), or temporarily configured (e.g., programmed) to operate in a certain manner or to perform certain operations described herein. Furthermore, as used herein, the phrase “hardware-implemented module” refers to a hardware module. Considering example embodiments in which hardware modules are temporarily configured (e.g., programmed), each of the hardware modules need not be configured or instantiated at any one instance in time. For example, where a hardware module includes a CPU configured by software to become a special-purpose processor, the CPU may be configured as respectively different special-purpose processors (e.g., each included in a different hardware module) at different times. Software (e.g., a software module) may accordingly configure one or more processors, for example, to become or otherwise constitute a particular hardware module at one instance of time and to become or otherwise constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory (e.g., a memory device) to which it is communicatively coupled. A further hardware module may then, at a later time, access the memory to retrieve and process the stored output. Hardware modules may also initiate communications with input or output devices, and can operate on a resource (e.g., a collection of information from a computing resource).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In some example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data.” “content,” “bits,” “values,” “elements.” “symbols.” “characters,” “terms,” “numbers.” “numerals,” or the like. These words, however, are merely convenient labels and are to be associated with appropriate physical quantities.

Unless specifically stated otherwise, discussions herein using words such as “accessing.” “processing,” “detecting.” “computing,” “calculating.” “determining.” “generating.” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The following enumerated descriptions describe various examples of methods, machine-readable media, and systems (e.g., machines, such as devices, or other apparatus) discussed herein. Any one or more features of an example, taken in isolation or combination, should be considered as being within the disclosure of this application.

A first example provides a method (e.g., a computer-implemented method) comprising: causing, by one or more processors, capture of an image that depicts an optically readable code exhibited by a measurement device that corresponds to a scheduled medical procedure; determining, by the one or more processors (and) based on the optically readable code depicted by the captured image, a hardware identifier of a network interface of the measurement device and a location identifier of the measurement device; identifying, by the one or more processors, the scheduled medical procedure based on the location identifier determined based on the optically readable code exhibited by the measurement device; and establishing, by the one or more processors, a network connection with the measurement device based on the determined hardware identifier of the network interface of the measurement device, the established network connection providing access to measurement data generated by the measurement device that corresponds to the scheduled medical procedure identified based on the location identifier of the measurement device.

A second example provides a method according to the first example, further comprising: extracting, from the optically readable code depicted by the captured image, a pairing trigger code; and wherein: the establishing of the network connection with the measurement device is based on (e.g., in response to) the extracting of the pairing trigger code from the optically readable code exhibited by the measurement device.

A third example provides a method according to the second example, wherein: the extracting of the pairing trigger code from the optically readable code from which the hardware identifier is extracted correlates the hardware identifier with the pairing trigger code; and the establishing of the network connection with the measurement device is based on (e.g., in response to) the extracted hardware identifier being correlated with the extracted pairing trigger code.

A fourth example provides a method according to any of the first through third examples, further comprising: extracting, from the optically readable code depicted by the captured image, a provider identifier of a provider of the measurement device; and wherein: the establishing of the network connection with the measurement device is based on (e.g., in response to) the extracting of the provider identifier of the provider of the measurement device from the optically readable code exhibited by the measurement device.

A fifth example provides a method according to any of the first through fourth examples, wherein: the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

A sixth example provides a method according to any of the first through fifth examples, further comprising: extracting, from the optically readable code depicted by the captured image, an app invocation code that corresponds to an app installed on a mobile device; and causing the mobile device to execute the app that corresponds to the app invocation code based on the extracting of the app invocation code from the optically readable code exhibited by the measurement device.

A seventh example provides a method according to the sixth example, wherein: the establishing of the network connection with the measurement device establishes the network connection between the mobile device caused to execute the app and the measurement device that exhibits the optically readable code.

An eighth example provides a method according to any of the first through seventh examples, wherein: the determining of the location identifier of the measurement device based on the optically readable code includes at least one of: extracting the location identifier from the optically readable code, or accessing a database that maps the location identifier to an intermediate identifier within the optically readable code.

A ninth example provides a machine-readable medium (e.g., a non-transitory machine-readable medium, such as a non-transitory machine-readable storage medium) comprising instructions that, when executed by one or more processors of a machine, cause the machine to perform operations comprising: causing capture of an image that depicts an optically readable code exhibited by a measurement device that corresponds to a scheduled medical procedure; determining, based on the optically readable code depicted by the captured image, a hardware identifier of a network interface of the measurement device and a location identifier of the measurement device; identifying the scheduled medical procedure based on the location identifier determined based on the optically readable code exhibited by the measurement device; and establishing a network connection with the measurement device based on the determined hardware identifier of the network interface of the measurement device, the established network connection providing access to measurement data generated by the measurement device that corresponds to the scheduled medical procedure identified based on the location identifier of the measurement device.

A tenth example provides a machine-readable medium according to the ninth example, wherein the operations further comprise: extracting, from the optically readable code depicted by the captured image, a pairing trigger code; and wherein: the establishing of the network connection with the measurement device based on (e.g., in response to) to the extracting of the pairing trigger code from the optically readable code exhibited by the measurement device.

An eleventh example provides a machine-readable medium according to the ninth example or the tenth example, wherein the operations further comprise: extracting, from the optically readable code depicted by the captured image, a provider identifier of a provider of the measurement device; and wherein: the establishing of the network connection with the measurement device is based on (e.g., in response to) the extracting of the provider identifier of the provider of the measurement device from the optically readable code exhibited by the measurement device.

A twelfth example provides a machine-readable medium according to any of the ninth through eleventh examples, wherein: the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

A thirteenth example provides a machine-readable medium according to any of the ninth through twelfth examples, wherein the operations further comprise: extracting, from the optically readable code depicted by the captured image, an app invocation code that corresponds to an app installed on a mobile device; and causing the mobile device to execute the app that corresponds to the app invocation code based on the extracting of the app invocation code from the optically readable code exhibited by the measurement device.

A fourteenth example provides a machine-readable medium according to any of the ninth through thirteenth examples, wherein: the establishing of the network connection with the measurement device establishes the network connection between the mobile device caused to execute the app and the measurement device that exhibits the optically readable code.

A fifteenth example provides a system (e.g., a computer system of one or more computers, or other system of one or more machines) comprising: one or more processors; and a memory storing instructions that, when executed by at least one processor among the one or more processors, cause the system to perform operations comprising: causing capture of an image that depicts an optically readable code exhibited by a measurement device that corresponds to a scheduled medical procedure; determining, based on the optically readable code depicted by the captured image, a hardware identifier of a network interface of the measurement device and a location identifier of the measurement device; identifying the scheduled medical procedure based on the location identifier determined based on the optically readable code exhibited by the measurement device; and establishing a network connection with the measurement device based on the determined hardware identifier of the network interface of the measurement device, the established network connection providing access to measurement data generated by the measurement device that corresponds to the scheduled medical procedure identified based on the location identifier of the measurement device.

A sixteenth example provides a system according to the fifteenth example, wherein the operations further comprise: extracting, from the optically readable code depicted by the captured image, a pairing trigger code; and wherein: the establishing of the network connection with the measurement device is based on (e.g., in response to) the extracting of the pairing trigger code from the optically readable code exhibited by the measurement device.

A seventeenth example provides a system according to the sixteenth example, wherein: the extracting of the pairing trigger code from the optically readable code from which the hardware identifier is extracted correlates the hardware identifier with the pairing trigger code; and the establishing of the network connection with the measurement device is based on (e.g., in response to) the extracted hardware identifier being correlated with the extracted pairing trigger code.

An eighteenth example provides a system according to any of the fifteenth through seventeenth examples, wherein the operations further comprise: extracting, from the optically readable code depicted by the captured image, a provider identifier of a provider of the measurement device; and wherein: the establishing of the network connection with the measurement device is based on (e.g., in response to) the extracting of the provider identifier of the provider of the measurement device from the optically readable code exhibited by the measurement device.

A nineteenth example provides a system according to any of the fifteenth through eighteenth examples, wherein: the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

A twentieth example provides system according to any of the fifteenth through nineteenth examples, wherein the operations further comprise: extracting, from the optically readable code depicted by the captured image, an app invocation code that corresponds to an app installed on a mobile device; and causing the mobile device to execute the app that corresponds to the app invocation code based on the extracting of the app invocation code from the optically readable code exhibited by the measurement device.

A twenty-first example provides a carrier medium carrying machine-readable instructions for controlling a machine to carry out the operations (e.g., method operations) performed in any one of the previously described examples.

1. A method comprising:
causing, by one or more processors, capture of an image that depicts an optically readable code exhibited by a measurement device that corresponds to a scheduled medical procedure;
determining, by the one or more processors based on the optically readable code depicted by the captured image, a hardware identifier of a network interface of the measurement device and a location identifier of the measurement device;
identifying, by the one or more processors, the scheduled medical procedure based on the location identifier determined based on the optically readable code exhibited by the measurement device; and
establishing, by the one or more processors, a network connection with the measurement device based on the determined hardware identifier of the network interface of the measurement device, the established network connection providing access to measurement data generated by the measurement device that corresponds to the scheduled medical procedure identified based on the location identifier of the measurement device.

2. The method of claim 1, further comprising:
extracting, from the optically readable code depicted by the captured image, a pairing trigger code; and wherein:
the establishing of the network connection with the measurement device is in response to the extracting of the pairing trigger code from the optically readable code exhibited by the measurement device.

3. The method of claim 2, wherein:
the extracting of the pairing trigger code from the optically readable code from which the hardware identifier is extracted correlates the hardware identifier with the pairing trigger code; and
the establishing of the network connection with the measurement device is based on the extracted hardware identifier being correlated with the extracted pairing trigger code.

4. The method of claim 1, further comprising:
extracting, from the optically readable code depicted by the captured image, a provider identifier of a provider of the measurement device; and wherein:
the establishing of the network connection with the measurement device is in response to the extracting of the provider identifier of the provider of the measurement device from the optically readable code exhibited by the measurement device.

5. The method of claim 1, wherein:
the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

6. The method of claim 1, further comprising:
extracting, from the optically readable code depicted by the captured image, an app invocation code that corresponds to an app installed on a mobile device; and
causing the mobile device to execute the app that corresponds to the app invocation code based on the extracting of the app invocation code from the optically readable code exhibited by the measurement device.

7. The method of claim 6, wherein:
the establishing of the network connection with the measurement device establishes the network connection between the mobile device caused to execute the app and the measurement device that exhibits the optically readable code.

8. The method of claim 1, wherein:
the determining of the location identifier of the measurement device based on the optically readable code includes at least one of extracting the location identifier from the optically readable code, or accessing a database that maps the location identifier to an intermediate identifier within the optically readable code.

9. A non-transitory machine-readable storage medium comprising instructions that, when executed by one or more processors of a machine, cause the machine to perform operations comprising:
causing capture of an image that depicts an optically readable code exhibited by a measurement device that corresponds to a scheduled medical procedure;
determining, based on the optically readable code depicted by the captured image, a hardware identifier of a network interface of the measurement device and a location identifier of the measurement device;
identifying the scheduled medical procedure based on the location identifier determined based on the optically readable code exhibited by the measurement device; and
establishing a network connection with the measurement device based on the determined hardware identifier of the network interface of the measurement device, the established network connection providing access to measurement data generated by the measurement device that corresponds to the scheduled medical procedure identified based on the location identifier of the measurement device.

10. The non-transitory machine-readable storage medium of claim 9, wherein the operations further comprise:
extracting, from the optically readable code depicted by the captured image, a pairing trigger code; and wherein:
the establishing of the network connection with the measurement device is in response to the extracting of the pairing trigger code from the optically readable code exhibited by the measurement device.

11. The non-transitory machine-readable storage medium of claim 9, wherein the operations further comprise:
extracting, from the optically readable code depicted by the captured image, a provider identifier of a provider of the measurement device; and wherein:
the establishing of the network connection with the measurement device is in response to the extracting of the provider identifier of the provider of the measurement device from the optically readable code exhibited by the measurement device.

12. The non-transitory machine-readable storage medium of claim 9, wherein: the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

13. The non-transitory machine-readable storage medium of claim 9, wherein the operations further comprise:
extracting, from the optically readable code depicted by the captured image, an app invocation code that corresponds to an app installed on a mobile device; and
causing the mobile device to execute the app that corresponds to the app invocation code based on the extracting of the app invocation code from the optically readable code exhibited by the measurement device.

14. The non-transitory machine-readable storage medium of claim 13, wherein: the establishing of the network connection with the measurement device establishes the network connection between the mobile device caused to execute the app and the measurement device that exhibits the optically readable code.

15. A system comprising:
one or more processors; and
a memory storing instructions that, when executed by at least one processor among the one or more processors, cause the system to perform operations comprising:
causing capture of an image that depicts an optically readable code exhibited by a measurement device that corresponds to a scheduled medical procedure;
determining, based on the optically readable code depicted by the captured image, a hardware identifier of a network interface of the measurement device and a location identifier of the measurement device;
identifying the scheduled medical procedure based on the location identifier determined based on the optically readable code exhibited by the measurement device; and
establishing a network connection with the measurement device based on the determined hardware identifier of the network interface of the measurement device, the established network connection providing access to measurement data generated by the measurement device that corresponds to the scheduled medical procedure identified based on the location identifier of the measurement device.

16. The system of claim 15, wherein the operations further comprise:
extracting, from the optically readable code depicted by the captured image, a pairing trigger code; and wherein:
the establishing of the network connection with the measurement device is in response to the extracting of the pairing trigger code from the optically readable code exhibited by the measurement device.

17. The system of claim 16, wherein:
the extracting of the pairing trigger code from the optically readable code from which the hardware identifier is extracted correlates the hardware identifier with the pairing trigger code; and
the establishing of the network connection with the measurement device is based on the extracted hardware identifier being correlated with the extracted pairing trigger code.

18. The system of claim 15, wherein the operations further comprise:
extracting, from the optically readable code depicted by the captured image, a provider identifier of a provider of the measurement device; and wherein:
the establishing of the network connection with the measurement device is in response to the extracting of the provider identifier of the provider of the measurement device from the optically readable code exhibited by the measurement device.

19. The system of claim 15, wherein:
the causing of the capture of the image that depicts the optically readable code exhibited by the measurement device includes prompting a user of a mobile device to initiate the establishing of the network connection with the measurement device by scanning the optically readable code exhibited by the measurement device via a camera of the mobile device.

20. The system of claim 15, wherein the operations further comprise:
extracting, from the optically readable code depicted by the captured image, an app invocation code that corresponds to an app installed on a mobile device; and
causing the mobile device to execute the app that corresponds to the app invocation code based on the extracting of the app invocation code from the optically readable code exhibited by the measurement device.
